(19) United States
(12) Patent Application Publication
(10) Pub. No.: US 2021/0215721 A1
(43) Pub. Date: Jul. 15, 2021

(54) Method and System for Identifying and Classifying a Liver Condition in a Human Subject

(71) Applicant: Roni Amiel, Orlando, FL (US)

(72) Inventor: Roni Amiel, Orlando, FL (US)

(21) Appl. No.: 16/743,195

(22) Filed: Jan. 15, 2020

(51) Int. Cl. G01N 33/72; G16H 50/20; G16H 10/40; G16H 50/30; G01N 33/92; G01N 33/68; G01N 33/573

(52) U.S. Cl. CPC G01N 33/728; G16H 50/20; G16H 10/40; G16H 50/30; G01N 2800/085; G01N 33/68; G01N 33/573; G01N 2800/60; G01N 33/92

(57) ABSTRACT

Identifying and classifying a liver condition in a human subject based on a blood sample obtained from the human subject in a system being functionally associated with at least one analyzer for analyzing the blood sample is disclosed. The system includes at least one of an input interface or a transceiver, one or more processors, and a computer readable storage medium for instructions execution by the processor(s). The storage medium has stored instructions to receive biographical information relating to the human subject, and instructions to receive, from the analyzer(s), measurements of a plurality of serum biomarkers. The storage medium further has stored instructions to apply a neural network algorithm to the received measurements and biographical information, and instructions to identify, based on an output of the neural network algorithm, the presence of a liver condition, and to classify a severity of the liver condition.

### FIELD OF THE DISCLOSED TECHNOLOGY

The disclosed technology relates generally to methods and systems for diagnosing a liver condition in a human subject, and, more specifically, to methods and systems for identifying the presence of a liver condition, and/or classifying the severity of such liver condition, using a blood sample.

### BACKGROUND OF THE DISCLOSED TECHNOLOGY

Chronic liver diseases, such as chronic viral hepatitis, non-alcoholic fatty liver diseases (NAFLD), and non-alcoholic steatohepatitis (NASH), usually develop over many years. These conditions are leading causes of morbidity and mortality globally, and the prevalence thereof has been increasing in recent years. NAFLD now represents the most common cause of abnormal liver blood tests and is the most common liver disease in the Western world. NASH is currently the second leading cause of liver disease among those waiting for liver transplants in the US.

Percutaneous liver biopsy remains the gold standard for precisely diagnosing NAFLD, specifying the category (severity) thereof. A biopsy is also necessary to assess the histopathologic criteria essential to diagnosis of NASH. Biopsies further allow for confirmation of steatosis, as well as determining a degree of lobular inflammation, ballooning, and fibrosis. However, such biopsies are invasive, and include an intrinsic risk factor to the procedure.

The severity of NAFLD is evaluated based on a scoring system, such as the NAFLD Activity Score, which evaluates, and assigns scores to, four domains: steatosis (scores in the range of 0-3), lobular inflammation (scores in the range of 0-3), hepatocyte ballooning (scores in the range of 0-2), and liver fibrosis (scores in the range of 0-4). In some cases, the first three scores are summed to generate an aggregate value, whereas the fibrosis staging score is maintained separate.

NASH is diagnosed based on an overall assessment of the biopsied tissue by a pathologist, using scoring systems such as the Steatosis, Activity, and Fibrosis (SAF) score. The SAF score evaluates the presence and extent of each individual component of steatosis, inflammation, and ballooning.

However, there remains a need in the art for a system and/or a method to diagnose and evaluate fatty liver diseases in a non-invasive and automated manner.

### SUMMARY OF THE DISCLOSED TECHNOLOGY

The present disclosure relates to a method and a system for non-invasively and accurately identifying the presence of a liver condition in a human subject, and classifying the severity of such a liver condition.

In accordance with an embodiment of the disclosed technology, there is provided a method of identifying and classifying a liver condition in a human subject. In accordance with the method, biographical information relating to the human subject is obtained. The biographical information includes at least age, gender, height, and weight. A plurality of analyzers are used to obtain, from serum or plasma in a blood sample obtained from the human subject, measurements of a plurality of serum biomarkers. The plurality of serum biomarkers include at least three biomarkers selected from the group consisting of Alpha-2-Macroglobulin, Apolipoprotein A1, Haptoglobin, total Bilirubin, gamma-glutamyl transpeptidase (GGT), alanine-aminotransferase (ALT), aspartate aminotransferase (AST), Total fasting cholesterol, fasting triglycerides, and fasting glucose. Using a processor executing instructions stored in a non-transitory computer memory, a neural network algorithm is applied to the measurements of the plurality of biomarkers and the biographical information. Based on an output of the neural network algorithm, the presence of a liver condition is identified, and the severity of the liver condition is classified.

In some embodiments, at least one test tube used for obtaining the blood sample is at least one of a test tube containing lithium heparin, a test tube containing a glycolytic inhibitor, a test tube containing sodium fluoride, and a test tube containing potassium oxalate.

In some embodiments, the output of the neural network algorithm includes a first score indicative of fibrosis of the liver of the human subject, a second score indicative of activity in the liver of the human subject, and a third score indicative of steatosis of the liver of the human subject. In some cases, the neural network algorithm is applied three times, to provide each of the first, second, and third scores, respectively.

In some embodiments, prior to the application of the neural network algorithm, at least some of the measurements of the plurality of serum biomarkers or at least one data item of the biographical information are pre-processed.

In some embodiments, the pre-processing includes logarithmically scaling at least some of the measurements of the plurality of serum biomarkers. In some such embodiments, a first subset of the measurements of the plurality of serum biomarkers are logarithmically scaled to compute the first score, a second subset of the measurements of the plurality of serum biomarkers is logarithmically scaled to compute the second score, and a third subset of the measurements of the plurality of serum biomarkers are logarithmically scaled to compute the third score.

In some embodiments, the pre-processing includes standardizing at least some of the measurements of the plurality of serum biomarkers and at least one data item of the biographical information. In some such embodiments, a first sub-group of the measurements of the plurality of serum biomarkers and the at least one data item are standardized to compute the first score, a second sub-group of the measurements of the plurality of serum biomarkers and the at least one data item are standardized to compute the second score, and third sub-group of the measurements of the plurality of serum biomarkers and the at least one data item are standardized to compute the third score.

In accordance with another embodiment of the disclosed technology, there is provided a system of identifying and classifying a liver condition in a human subject based on a blood sample obtained from the human subject, the system being functionally associated with at least one analyzer for analyzing the blood sample. The system includes at least one of an input interface or a transceiver, one or more processors functionally associated with the at least one input interface or transceiver, and a non-transitory computer readable storage medium for instructions execution by the one or more processors. The non-transitory computer readable storage medium has stored instructions to receive biographical information relating to the human subject, including at least age, gender, height, and weight. The storage medium further has stored instructions to receive, from the at least one analyzer, measurements of a plurality of serum biomarkers, the plurality of serum biomarkers including at least three biomarkers selected from the group consisting of Alpha-2-Macroglobulin, Apolipoprotein A1, Haptoglobin, total Bilirubin, gamma-glutamyl transpeptidase (GGT), alanine-aminotransferase (ALT), aspartate aminotransferase (AST), Total fasting cholesterol, fasting triglycerides, and fasting glucose. Further stored instructions are to apply a neural network algorithm to the measurements of the plurality of biomarkers and the biographical information. The storage medium has stored additional instructions to identify, based on an output of the neural network algorithm, the presence of a liver condition, and to classify a severity of the liver condition.

In some embodiments, the instructions to apply the neural network algorithm include instructions to provide, as output of the neural network algorithm, a first score indicative of fibrosis of the liver of the human subject, a second score indicative of activity in the liver of the human subject, and a third score indicative of steatosis of the liver of the human subject.

In some such embodiments, the instructions to apply the neural network algorithm include instructions to apply the neural network algorithm three times, to provide each of the first, second, and third scores, respectively.

In some embodiments, the non-transitory computer readable storage medium further has stored instructions, to be executed prior to execution of the instructions to applying the neural network algorithm, to pre-process at least some of the measurements of the plurality of serum biomarkers or at least one data item of the biographical information.

In some embodiments, the instructions to pre-process include instructions to logarithmically scale at least some of the measurements of the plurality of serum biomarkers. In some such embodiments, the instructions to logarithmically scale include instructions to logarithmically scale a first subset of the measurements of the plurality of serum biomarkers to compute the first score, a second subset of the measurements of the plurality of serum biomarkers to compute the second score, and third subset of the measurements of the plurality of serum biomarkers to compute the third score.

In some embodiments, the instructions to pre-process include instructions to standardize at least some of the measurements of the plurality of serum biomarkers and at least one data item of the biographical information. In some such embodiments, the instructions to standardize include instructions to standardize a first sub-group of the measurements of the plurality of serum biomarkers and the at least one data item to compute the first score, a second sub-group of the measurements of the plurality of serum biomarkers and the at least one data item to compute the second score, and third sub-group of the measurements of the plurality of serum biomarkers and the at least one data item to compute the third score.

### BRIEF DESCRIPTION OF THE DRAWINGS

FIG. 1 is a schematic illustration of a system for identifying the presence of a liver condition, and classifying a severity thereof, according to an embodiment of the disclosed technology.

FIG. 2 is a flow-chart of a method for identifying the presence of a liver condition, and classifying a severity thereof, according to an embodiment of the disclosed technology.

FIG. 3A is a table representation of different serum biomarker measurements and biographical information used to identify the presence of different aspects of liver condition, according to an embodiment of the disclosed technology.

FIGS. 3B, 3C, AND 4 are table representations of a correlation between assigned scores, and severity of liver condition, for three measured parameters, according to an embodiment of the disclosed technology.

FIG. 5 shows a high-level block diagram of a device that may be used to carry out the disclosed technology.

### DETAILED DESCRIPTION OF EMBODIMENTS OF THE DISCLOSED TECHNOLOGY

In an embodiment of the disclosed technology, a system and a corresponding method of identifying and classifying a liver condition in a human subject based on a blood sample obtained from the human subject, the system being functionally associated with at least one analyzer for analyzing the blood sample. The system includes at least one of an input interface or a transceiver, one or more processors, and a computer readable storage medium for instructions execution by the processor(s). The storage medium has stored instructions to receive biographical information relating to the human subject, and instructions to receive, from the analyzer(s), measurements of a plurality of serum biomarkers. The storage medium further has stored instructions to apply a neural network algorithm to the received measurements and biographical information, and instructions to identify, based on an output of the neural network algorithm, the presence of a liver condition, and to classify a severity of the liver condition.

Embodiments of the disclosed technology will become clearer in view of the following description of the drawings.

In the context of the present specification and claims, the term “Fibrosis of the liver” is defined as formation of excess fibrous connective tissue in the liver or the formation of an abnormally large amount of scar tissue in the liver.

In the context of the present specification and claims, the term “Activity of the liver” is defined as inflammation of the liver or hepatic necroinflammatory activity.

In the context of the present specification and claims, the term “Steatosis of the liver” is defined as abnormal retention or accumulation of fat in the liver.

In the context of the present specification and claims, the term “approximately” is defined as being within 10% of a target number or measure.

It should be understood that the use of “and/or” is defined inclusively such that the term “a and/or b” should be read to include the sets: “a and b,” “a or b,” “a,” “b.”

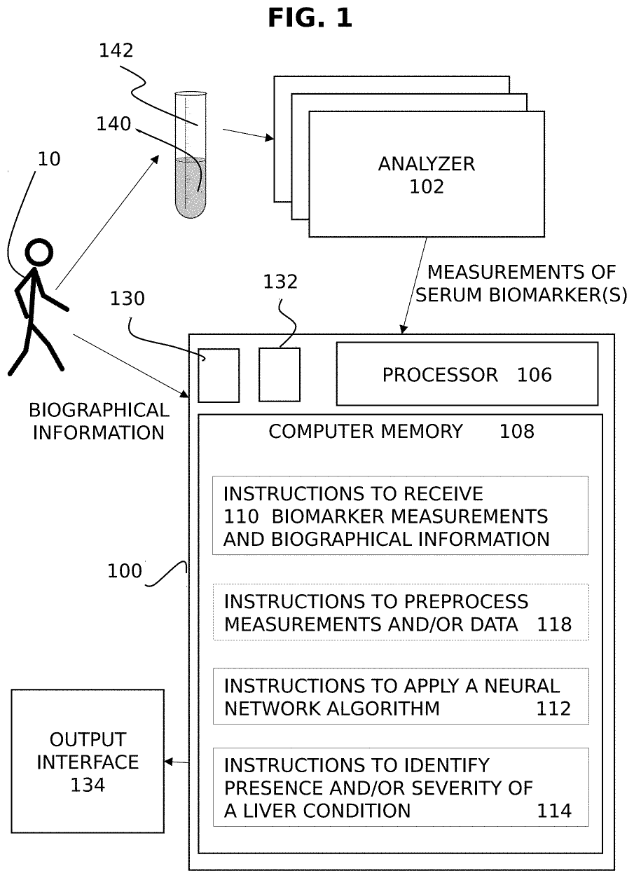

Reference is now made to FIG. 1, which is a schematic illustration of a system for identifying the presence of a liver condition, and classifying a severity thereof, according to an embodiment of the disclosed technology. As seen, a system 100 according to the disclosed technology comprises a computing device, functionally associated with a plurality of analyzers 102. The system 100 includes one or more processors 106, and a non-transitory computer readable storage medium 108, which stores instructions to be executed by the processor(s) 106.

Computer readable storage medium 108 has stored:
instructions 110 to receive, from analyzers 102, measurements of serum biomarkers obtained from blood of a human subject 10, as well as biographical information of the human subject 10;
instructions 112 to apply a neural network algorithm to the measurements of serum biomarkers and to the biographical information of the human subject; and
instructions 114 to identify, based on the output of the neural network algorithm, the presence or absence of a liver condition, and if a liver condition is present, to classify the severity of the liver condition.

In some embodiments, computer readable storage medium 108 further has stored instructions 118 to scale at least some of the measurements of serum biomarkers, and/or instructions to standardize at least some of the measurements of serum biomarkers and/or at least some of the biographical information.

The instructions 110 to receive measurements of serum biomarkers include instructions to receive measurements of at least three, at least four, at least five, or all of the following biomarkers:
Alpha-2-Macroglobulin;
Apolipoprotein A1;
Haptoglobin;
total Bilirubin;
gamma-glutamyl transpeptidase (GGT);
alanine-aminotransferase (ALT);
aspartate aminotransferase (AST);
Total fasting cholesterol;
fasting triglycerides; and
fasting glucose.

The instructions 110 to receive measurements of serum biomarkers include instructions to receive at least the human subject's age, gender, height, and weight.

In some embodiments, system 100 further includes, or is functionally associated with, at least one input interface 130, such as a keyboard, touchscreen, touchpad, mouse, and the like, functionally associated with processor 106. A user, such as a medical practitioner, may use the input interface(s) 130 to provide at least some of the data received when executing instructions 110, such as at least some of the biographical information.

In some embodiments, system 100 further includes at least one transceiver 132, functionally associated with processor 106, and adapted for communication with the analyzer(s) 102 and/or with other devices adapted to provide the inputs received by execution of instructions 110, such as a scale.

In some embodiments, instructions 114 to identify the presence of a liver condition, and, if relevant, to classify the severity of the liver condition, further include instructions to provide an output including the identified presence and/or severity of the liver condition. In some such embodiments, system 100 further includes, or is functionally associated with, an output interface 134, such as a display screen or a speaker, via which the output is visually or audibly provided to a user, such as a medical practitioner. In some embodiments, the output may be provided in an electronic communication, such as an e-mail message, for example via transceiver 132.

Typically, a blood sample 140, used to extract the measurements of the serum biomarkers received system 100, is collected from the human subject using test tubes 142 as known in the art. In some embodiments, the blood sample must have sufficient volume to provide a 500 microliter volume of plasma or serum, after centrifugation thereof. In some embodiments, the blood sample must have sufficient volume to provide a 200 microliter volume of plasma or serum, after centrifugation thereof.

In some embodiments, at least one test tube 142 is a test tube containing lithium heparin. In some embodiments, at least one test tube 142 is a test tube containing a glycolytic inhibitor, such as sodium fluoride or potassium oxalate, required for extracting a fasting glucose biomarker measurement.

In some embodiments, the analyzer(s) 102 include an analyzer adapted to carry out a Nephelometry analysis to identify biomarker measurements for at least some biomarkers, such as Alpha-2-macroglobulin, Haptoglobin, and apolipoprotein A1. Examples of suitable analyzers include Siemens Healthcare Diagnostics models BN1 or BN Prospec Vista, commercially available from Siemens® AG of Munich, Germany and Immage 800 commercially available from Beckman Coulter® of Indianapolis, Ind., USA.

In some embodiments, the analyzer(s) 102 include an analyzer adapted to carry out a Turbidimetry analysis to identify biomarker measurements for at least some biomarkers, such as Alpha-2-macroglobulin, Haptoglobin, and apolipoprotein A1. Examples of suitable analyzers include Siemens Healthcare Diagnostics models Dimension-RXL, ArX, XP, Atelica® CH 930, or Advia 1650, commercially available from Siemens® AG of Munich, Germany; models AU400, AU600, AU640, or AU2700 commercially available from Beckman Coulter® of Indianapolis, Ind., USA; and models T20, T20XT, T30, T60, and T60 new generation commercially available from Thermo Fisher Scientific® of Waltham, Mass., USA.

In some embodiments, the analyzer(s) 102 include an analyzers adapted to extract at least one GGT measurements, total bilirubin measurements, and/or ALT measurements. Examples of suitable analyzers include Siemens Healthcare Diagnostics models Dimension-RXL, ArX, XPAND Vista, Atelica® CH 930, or Advia 1650, commercially available from Siemens® AG of Munich, Germany; models AU400, AU600, AU640, or AU2700 commercially available from Beckman Coulter® of Indianapolis, Ind., USA; models T20, T20XT, T30, T60, and T60 new generation commercially available from Thermo Fisher Scientific® of Waltham, Mass., USA; and Hitachi 917, Modular P, Integra 400, Cobas 6000, or Cobas 8000 commercially available from Roche Diagnostics® of Basel, Switzerland.

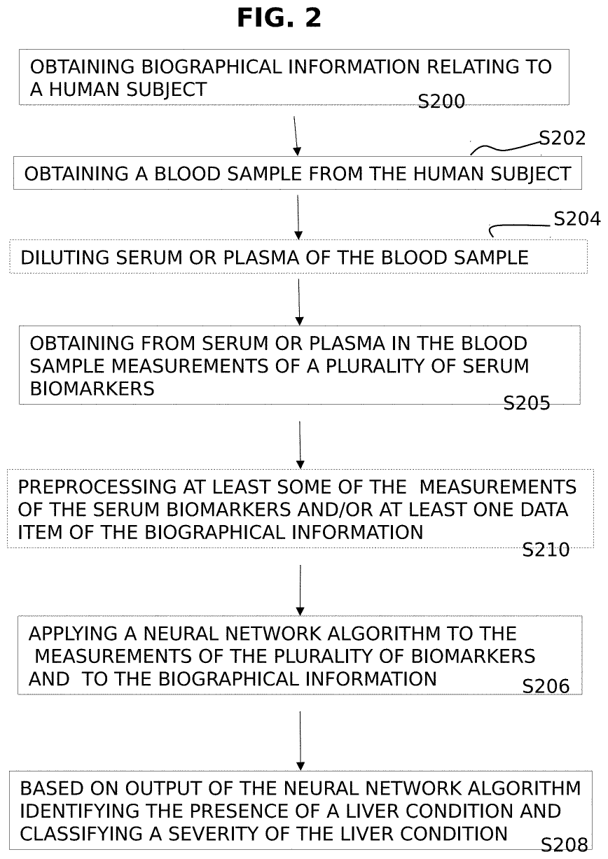

Reference is now additionally made to FIG. 2, which is a flow-chart of a method for identifying the presence of a liver condition, and classifying a severity thereof, according to an embodiment of the disclosed technology. The method of FIG. 2 is described herein with respect to the system of FIG. 1. However, any other suitable system may be used to implement the method of FIG. 2.

As seen in FIG. 2, at step S200, biographical information relating to a human subject, such as subject 10 of FIG. 1, is obtained, for example by processor 106 executing instructions 110. In some embodiments, the biographical information includes at least the subject's age, gender, height, and weight. At step S202, which may occur before, after, or concurrently with step S200, a blood sample of the human subject is obtained. As discussed hereinabove with respect to FIG. 1, the blood sample is typically obtained in test tubes, using methods known in the art. In some embodiments, specific types of test tubes are used, as discussed hereinabove with respect to FIG. 1. Serum or plasma is then extracted from the blood sample, using methods known in the art, such as centrifugation.

In some embodiments, the obtained blood sample may optionally be diluted, or otherwise preprocessed, at step S204. This may occur when the blood sample includes components which would interfere with the remainder of the method, such as lipids.

At step S205, measurements of a plurality of serum biomarkers are obtained from the serum or plasma in the blood sample, for example by processor 106 executing instructions 110. The measurements are computed by one or more analyzers, such as analyzers 102 of FIG. 1, and are received from the analyzer(s). The obtained measurements include measurements for at least three, at least four, and preferably all of:
Alpha-2-Macroglobulin;
Apolipoprotein A1;
Haptoglobin;
total Bilirubin;
gamma-glutamyl transpeptidase (GGT);
alanine-aminotransferase (ALT);
aspartate aminotransferase (AST);
Total fasting cholesterol;
fasting triglycerides; and
fasting glucose.

At step S206, a neural network algorithm is applied to at least some of, or to all of, the measurements of the plurality of biomarkers and to at least some of, or to all of, the obtained biographical information, for example by processor 106 executing instructions 112. Based on the output of the neural network algorithm, at step S208, the presence or absence of a liver condition is identified, and, in cases in which such liver condition is identified, its severity is classified, for example by processor 106 executing instructions 114.

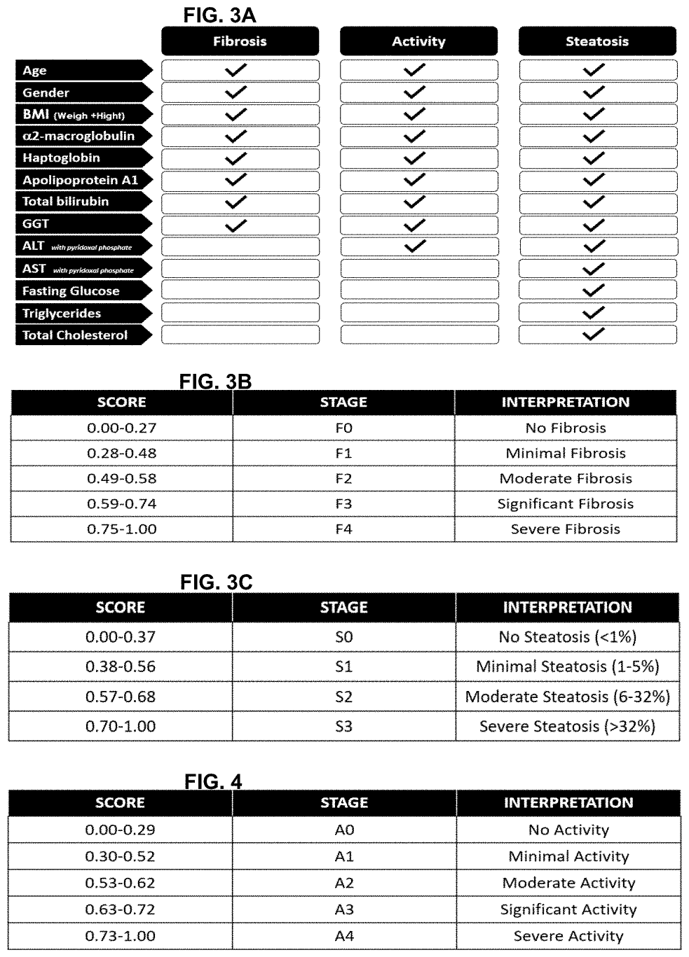

In some embodiments, the application of the neural network algorithm at step S206 and/or the identification of a liver condition at step S208 is divided into processes for identification of at least one of three aspects, including Fibrosis of the liver, Activity, and Steatosis. In some embodiments, different ones of the obtained serum biomarker measurements and biographical information data items are applied to the neural network algorithm, and thus are used to evaluate, or identify, the presence or absence of the different aspects of a liver condition, as demonstrated in FIG. 3.

In some embodiments, the classification of severity of the liver condition is divided into classification of at least one of the three aspects. In some such embodiments, the classification is dependent on a score obtained for at least one of, or each of, the three aspects, the score being a continuous score in the range of 0-1. The correlation between assigned scores and the severity of Fibrosis, Activity, and Steatosis, are presented in FIGS. 3B, 3C, and 4, respectively.

In some embodiments, prior to application of the neural network algorithm at step S206, at least some of the serum biomarker measurements and/or some biographical information data items preprocessed, for example by processor 106 executing instructions 118. In some such embodiments, the values used for the neural network algorithm, at step S206, are the preprocessed values resulting from step S210. In some such embodiments, the preprocessing includes logarithmically scaling at least some of the serum biomarker measurements, for example using logarithmic base 10. In some embodiments, the preprocessing includes standardizing values of at least some of the serum biomarker measurements and/or of at least some biographical information data items.

In some embodiments, in which the preprocessing includes logarithmic scaling of serum biomarker measurements, the specific serum biomarker measurements being scaled are dependent on the aspect of liver condition being evaluated. In some embodiments, for the evaluation of Fibrosis, scaling at step S210 includes logarithmically scaling measurements of alpha-2 macroglobulin, total bilirubin, haptoglobin, and GGT. In some embodiments, for the evaluation of Activity, scaling at step S210 includes logarithmically scaling measurements of alpha-2 macroglobulin, ALT total bilirubin, haptoglobin, and GGT. In some embodiments, for the evaluation of Steatosis, scaling at step S210 includes logarithmically scaling measurements of alpha-2 macroglobulin, ALT, AST, total bilirubin, total cholesterol, GGT, fasting glucose, haptoglobin, and triglycerides.

In some embodiments, in which the preprocessing includes standardizing of serum biomarker measurements and/or of biographical information data items, the specific serum biomarker measurements and/or biographical information data items being standardized are dependent on the aspect of liver condition being evaluated. In some embodiments, for the evaluation of Fibrosis, standardizing at step S210 includes standardizing of age, alpha-2 macroglobulin measurement, haptoglobin measurement, apolipoprotein A1 measurement, total bilirubin measurement, and GGT measurement. In some embodiments, for the evaluation of Activity, standardizing at step S210 includes standardizing of age, alpha-2 macroglobulin measurement, haptoglobin measurement, apolipoprotein A1 measurement, total bilirubin measurement, GGT measurement, and ALT measurement. In some embodiments, for the evaluation of Steatosis, standardizing at step S210 includes standardizing of age, alpha-2 macroglobulin measurement, haptoglobin measurement, apolipoprotein A1 measurement, total bilirubin measurement, GGT measurement, ALT, AST measurement, BMI, fasting blood glucose measurement, triglycerides measurement, and total cholesterol measurement.

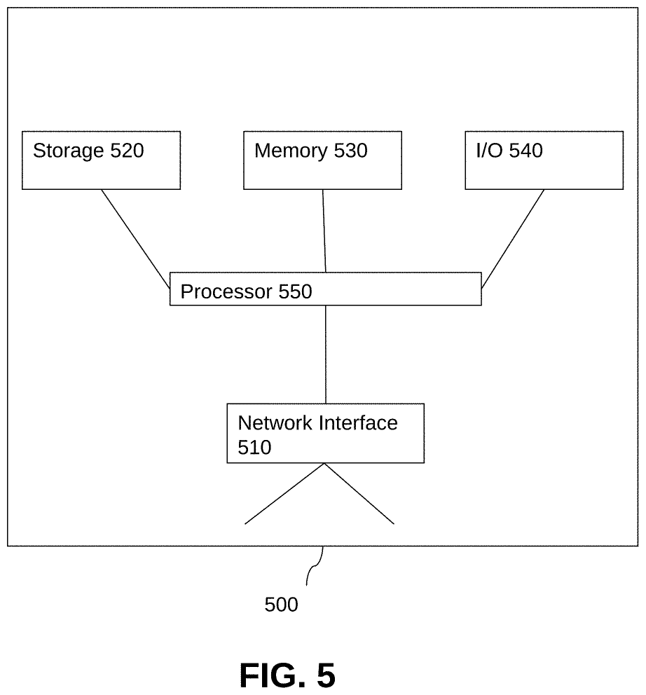

FIG. 5 shows a high-level block diagram of a device that may be used to carry out the disclosed technology. Device 500 comprises a processor 550 that controls the overall operation of the computer by executing the device's program instructions which define such operation. The device's program instructions may be stored in a storage device 520 (e.g., magnetic disk, database) and loaded into memory 530 when execution of the console's program instructions is desired. Thus, the device's operation will be defined by the device's program instructions stored in memory 530 and/or storage 520, and the console will be controlled by processor 550 executing the console's program instructions. A device 500 also includes one or a plurality of input network interfaces for communicating with other devices via a network (e.g., the internet). The device 500 further includes an electrical input interface. A device 500 also includes one or more output network interfaces 510 for communicating with other devices. Device 500 also includes input/output 540 representing devices which allow for user interaction with a computer (e.g., display, keyboard, mouse, speakers, buttons, etc.). One skilled in the art will recognize that an implementation of an actual device will contain other components as well, and that FIG. 5 is a high level representation of some of the components of such a device for illustrative purposes. It should also be understood by one skilled in the art that the method and devices depicted in FIGS. 1 and 2 may be implemented using a device such as is shown in FIG. 5.

For purposes of this disclosure, the term “substantially” is defined as “at least 95% of the term which it modifies.

Any device or aspect of the technology can “comprise” or “consist of” the item it modifies, whether explicitly written as such or otherwise.

When the term “or” is used, it creates a group which has within either term being connected by the conjunction as well as both terms being connected by the conjunction.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods and apparatuses described hereinabove are also contemplated and within the scope of the invention.

1. A method of identifying and classifying a liver condition in a human subject, the method comprising:
obtaining biographical information relating to the human subject, said biographical information including at least age, gender, height, and weight;
using a plurality of analyzers, obtaining from serum or plasma in a blood sample obtained from the human subject measurements of a plurality of serum biomarkers, said plurality of serum biomarkers including at least three biomarkers selected from the group consisting of Alpha-2-Macroglobulin, Apolipoprotein A1, Haptoglobin, total Bilirubin, gamma-glutamyl transpeptidase (GGT), alanine-aminotransferase (ALT), aspartate aminotransferase (AST), Total fasting cholesterol, fasting triglycerides, and fasting glucose;
using a processor executing instructions stored in a non-transitory computer memory, applying a neural network algorithm to said measurements of said plurality of biomarkers and said biographical information; and
based on an output of said neural network algorithm, identifying the presence of a liver condition and classifying a severity of said liver condition.

2. The method of claim 1, wherein at least one test tube used for obtaining said blood sample comprises at least one of a test tube containing lithium heparin, a test tube containing a glycolytic inhibitor, a test tube containing sodium fluoride, and a test tube containing potassium oxalate.

3. The method of claim 1, wherein said output of said neural network algorithm includes a first score indicative of fibrosis of the liver of the human subject, a second score indicative of activity in the liver of the human subject, and a third score indicative of steatosis of the liver of the human subject.

4. The method of claim 3, wherein said neural network algorithm is applied three times, to provide said first, second, and third scores, respectively.

5. The method of claim 1, further comprising, prior to said applying said neural network algorithm, pre-processing at least some of said measurements of said plurality of serum biomarkers or at least one data item of said biographical information.

6. The method of claim 5, wherein said pre-processing comprises logarithmically scaling at least some of said measurements of said plurality of serum biomarkers.

7. The method of claim 6, wherein said output of said neural network algorithm includes a first score indicative of fibrosis of the liver of the human subject, a second score indicative of activity in the liver of the human subject, and a third score indicative of steatosis of the liver of the human subject, and wherein said logarithmically scaling comprises logarithmically scaling a first subset of said measurements of said plurality of serum biomarkers to compute said first score, a second subset of said measurements of said plurality of serum biomarkers to compute said second score, and third subset of said measurements of said plurality of serum biomarkers to compute said third score.

8. The method of claim 5, wherein said pre-processing comprises standardizing at least some of said measurements of said plurality of serum biomarkers and at least one data item of said biographical information.

9. The method of claim 8, wherein said output of said neural network algorithm includes a first score indicative of fibrosis of the liver of the human subject, a second score indicative of activity in the liver of the human subject, and a third score indicative of steatosis of the liver of the human subject, and wherein said standardizing comprises standardizing a first sub-group of said measurements of said plurality of serum biomarkers and said at least one data item to compute said first score, a second sub-group of said measurements of said plurality of serum biomarkers and said at least one data item to compute said second score, and third sub-group of said measurements of said plurality of serum biomarkers and said at least one data item to compute said third score.

10. A system of identifying and classifying a liver condition in a human subject based on a blood sample obtained from the human subject, the system being functionally associated with at least one analyzer for analyzing the blood sample, the system comprising:
at least one of an input interface or a transceiver;
one or more processors functionally associated with said at least one input interface or transceiver; and
a non-transitory computer readable storage medium for instructions execution by the one or more processors, the non-transitory computer readable storage medium having stored:
instructions to receive biographical information relating to the human subject, said biographical information including at least age, gender, height, and weight;
instructions to receive, from said at least one analyzer, measurements of a plurality of serum biomarkers, said plurality of serum biomarkers including at least three biomarkers selected from the group consisting of Alpha-2-Macroglobulin, Apolipoprotein A1, Haptoglobin, total Bilirubin, gamma-glutamyl transpeptidase (GGT), alanine-aminotransferase (ALT), aspartate aminotransferase (AST), Total fasting cholesterol, fasting triglycerides, and fasting glucose;
instructions to apply a neural network algorithm to said measurements of said plurality of biomarkers and said biographical information; and
instructions to identify, based on an output of said neural network algorithm, the presence of a liver condition, and to classify a severity of said liver condition.

11. The system of claim 10, wherein said instructions to apply said neural network algorithm comprise instructions to provide, as output of said neural network algorithm, a first score indicative of fibrosis of the liver of the human subject, a second score indicative of activity in the liver of the human subject, and a third score indicative of steatosis of the liver of the human subject.

12. The system of claim 11, wherein said instructions to apply said neural network algorithm comprise instructions to apply said neural network algorithm three times, to provide each of said first, second, and third scores, respectively.

13. The system of claim 10, wherein said non-transitory computer readable storage medium further has stored instructions, to be executed prior to execution of said instructions to applying said neural network algorithm, to pre-process at least some of said measurements of said plurality of serum biomarkers or at least one data item of said biographical information.

14. The system of claim 13, wherein said instructions to pre-process comprise instructions to logarithmically scale at least some of said measurements of said plurality of serum biomarkers.

15. The system of claim 14, wherein said instructions to apply said neural network algorithm comprise instructions to provide a first score indicative of fibrosis of the liver of the human subject, a second score indicative of activity in the liver of the human subject, and a third score indicative of steatosis of the liver of the human subject, and wherein said instructions to logarithmically scale comprise instructions to logarithmically scale a first subset of said measurements of said plurality of serum biomarkers to compute said first score, a second subset of said measurements of said plurality of serum biomarkers to compute said second score, and third subset of said measurements of said plurality of serum biomarkers to compute said third score.

16. The system of claim 13, wherein said instructions to pre-process comprise instructions to standardize at least some of said measurements of said plurality of serum biomarkers and at least one data item of said biographical information.

17. The system of claim 16, wherein said instructions to apply said neural network algorithm comprise instructions to provide a first score indicative of fibrosis of the liver of the human subject, a second score indicative of activity in the liver of the human subject, and a third score indicative of steatosis of the liver of the human subject, and wherein said instructions to standardize comprise instructions to standardize a first sub-group of said measurements of said plurality of serum biomarkers and said at least one data item to compute said first score, a second sub-group of said measurements of said plurality of serum biomarkers and said at least one data item to compute said second score, and third sub-group of said measurements of said plurality of serum biomarkers and said at least one data item to compute said third score.
